(19) United States
(12) Patent Application Publication
(10) Pub. No.: US 2025/0084370 A1
(43) Pub. Date: Mar. 13, 2025

(54) METHODS FOR SELECTIVELY REDUCING IMMUNOGENICITY IN A TRANSPLANT

(71) Applicants: TRANSIMMUNE AG, Düsseldorf (DE); YALE UNIVERSITY, New Haven, CT (US)

(72) Inventors: Richard EDELSON, New Haven, CT (US); Olga SOBOLEV, Düsseldorf (DE); Douglas HANLON, New Haven, CT (US); Aaron VASSAL, New Haven, CT (US); Kazuki TATSUNO, New Haven, CT (US); Patrick HAN, New Haven, CT (US)

(73) Assignees: TRANSIMMUNE AG, Düsseldorf (DE); YALE UNIVERSITY, New Haven, CT (US)

(21) Appl. No.: 18/580,098

(22) PCT Filed: Jul. 29, 2022
(86) PCT No.: PCT/EP2022/071418

§ 371 Date: Jan. 17, 2024

Related U.S. Application Data
(60) Provisional application No. 63/203,727, filed on Jul. 29, 2021.

(30) Foreign Application Priority Data
Sep. 10, 2021 (EP) 21196100.8

(51) Int. Cl. C12N 5/07; A61K 35/12; A61P 37/06; C12N 13/00

(52) U.S. Cl. CPC C12N 5/06; A61K 35/12; A61P 37/06; C12N 13/00; C12N 2501/999; C12N 2502/1114; C12N 2502/1121; C12N 2502/1178

(57) ABSTRACT

The present invention relates to methods for reducing or eliminating reactive T cells from a transplant or a part thereof prior to transplantation. The present invention also relates to methods for reducing immunogenicity in a transplant or a part thereof prior to transplantation. The present invention further relates to transplants obtained by the described methods and apoptotic agent treated transplants for use in reducing or preventing inflammatory conditions such as graft-versus-host disease. Specifically, the methods can be used to reduce graft versus host disease following transplantation.

### FIELD OF THE INVENTION

The present invention relates to methods for reducing or eliminating reactive T cells from a transplant or a part thereof prior to transplantation. The present invention also relates to methods for reducing immunogenicity in a transplant or a part thereof prior to transplantation. The present invention further relates to transplants obtained by the described methods and apoptotic agent treated transplants for use in reducing or preventing inflammatory conditions such as graft-versus-host disease. Specifically, the methods can be used to reduce graft versus host disease.

### BACKGROUND OF THE INVENTION

Transplantation of organs, tissue or cells from one genetically distinct person (donor) to another (recipient) is hindered by the recipient's immunologic rejection of the donated organs or cells. This rejection phenomenon is understood to involve both cellular and humoral mechanisms, mediated respectively by T cells and anti bodies. In particular, hematopoietic stem cell transplants, which are transplants of blood cells or bone marrow from the same individual (an autologous transplant, or autograft) or different individual (an allogeneic transplant, or allograft), are of proven benefit in treating a variety of immune dysfunctions and malignancies. However, the widespread application of allogeneic transplant procedures is restricted by the availability of suitable donors. A suitable allogeneic donor is an individual with an identical or near identical profile of cell-surface antigens known as major histocompatibility antigens (MHC) or HLA antigens. There are many alternative forms (alleles) of each of the HLA antigens, and thus the chance of two unrelated individuals being closely HLA-matched is extremely small. As HLA antigen loci are closely linked, an individual inherits the HLA alleles as two sets, one from each parent. Therefore, a certain percentage of individuals eligible for an allogeneic stem cell transplant will have an HLA-identical relative. Nearly all eligible patients will have a relative who is haploidentical, sharing half of the HLA antigens with the patient.

One of the major barriers to successful transplantation between HLA-mismatched or haploidentical individuals is the risk of developing potentially life-threatening graft-versus-host disease (GvHD). GvHD occurs when donor T cells recognize the tissues of the recipient as foreign, proliferate to form many copies of themselves in the recipient and attack normal cells of the recipient, causing a severe inflammatory disease. The activity of donor T cells against HLA antigens, and other minor histocompatibility antigens, on the surface of recipient cells is at the center of GvHD pathology, and approaches to date to control GvHD have employed in vivo or ex vivo methods that suppress the function of T cells. The majority of efforts have focused on removing or suppressing the activity of the entire repertoire of donor T cells, or major subsets of these, resulting in global immunosuppression in the recipient. However, donor T cells, while contributing to the development of GvHD, also contribute positively to transplant outcome by facilitating engraftment and controlling opportunistic infections. Donor T cells also are associated with a beneficial “graft-versus-tumor” effect. Thus, indiscriminate depletion of T cells from the allograft, while reducing GvHD, also increases graft failure, susceptibility to opportunistic infections, and leukemia relapse rate.

Thus, there remains a need for selectively reducing or eliminating reactive T cells from a transplant. Put in other words, there remains a need for methods which are able to selectively reduce immunogenicity of a transplant. Such methods would allow allo-antigen reactive T cells to be effectively reduced or removed from an allograft, and thus improve the efficacy of allografts for the treatment of autoimmune diseases.

### OBJECTIVES AND SUMMARY OF THE INVENTION

One objective of the present invention is to provide methods for selectively reducing immunogenicity in a transplant or a part thereof prior to transplantation.

Another objective of the present invention is to provide methods for reducing reactive immune cells, in particular T cells, in a transplant or a part thereof prior to transplantation.

Another objective of the present invention is to provide a transplant or a part thereof for use in preventing or reducing autoimmune diseases, specifically GvHD.

It is still another objective to provide methods of treating autoimmune diseases, specifically GvHD.

These and other objectives as they will become apparent from the ensuing description hereinafter are solved by the subject matter of the independent claims. Some of the preferred embodiments of the present invention form the subject matter of the dependent claims. Yet other embodiments of the present invention may be taken from the ensuing description.

The present invention as illustratively described in the following may suitably be practiced in the absence of any element or elements, limitation or limitations, not specifically disclosed herein.

The present invention is described with respect to particular embodiments below and with reference to certain figures but the invention is not limited thereto but only by the claims.

### First Aspect: A Method to Selectively Reduce Immunogenicity in a Transplant Using a Recipient Tissue Sample

In a first aspect, the invention relates to a method comprising the following steps:
a) Providing a transplant or a part thereof from a donor;
b) Providing a tissue sample from a recipient comprising cells capable of presenting antigens;
c) Exposing the tissue sample of step b) to at least one anti-proliferative agent, at least one apoptotic agent, lethal radiation or at least one cycle of freeze-thawing;
d) Combining the transplant or part thereof of step a) with the exposed tissue sample of step c); and
e) Exposing the combination of step d) to a low dose of at least one apoptotic agent.

In one embodiment, the method is performed prior to transplantation.

In one embodiment the method is for selectively reducing immunogenicity in a transplant or a part thereof prior to transplantation.

In one embodiment, the method is for selectively reducing or eliminating reactive immune cells from the transplant prior to transplantation. Preferably, the method is for selectively reducing or eliminating reactive T cells from the transplant prior to transplantation.

In one embodiment, the method is for selectively reducing or eliminating reactive donor T cells from the transplant prior to transplantation.

In one embodiment, the method is for selectively reducing immunogenicity in a transplant or a part thereof prior to transplantation and selectively reducing or eliminating reactive donor T cells from the transplant prior to transplantation.

In one embodiment, the method may also refer to selectively reducing immunoreactivity caused by a graft (GvHD), including reducing immunogenicity of the recipient against the graft. For example, in the process of graft rejection, antigen reactive lymphocytes from the recipient proliferate in response to the donor's allo-antigens. This may take place outside the actual graft, e.g. in the recipient lymphnodes. The recipient's sensitized effector cells can then mediate immune destruction of the graft leading to inflammatory processes throughout the body, possibly affecting organs such as e.g. the skin, gastrointestinal tract and liver. Thus, this embodiment includes immunoreactivity outside the transplant but caused by the transplant. One beneficial effect of the methods as described herein resides inter alia in the reduction of immunoreactivity outside the graft once it has been transplanted.

The following embodiments and explanations relate to the selective reduction of immunogenicity in a transplant or a part thereof and/or the reduction or elimination of reactive T cells or reactive donor T cells. It needs to be understood that all method steps may be performed in vitro.

In one embodiment of the first aspect, the invention relates to a method comprising the following steps:
a) Providing a transplant or a part thereof from a donor;
b) Providing a tissue sample from a recipient comprising cells capable of presenting antigens;
c) Exposing the tissue sample of step b) to at least one anti-proliferative agent, at least one apoptotic agent, lethal radiation or at least one cycle of freeze-thawing;
d) Combining the transplant or part thereof of step a) with the exposed tissue sample of step c);
d1) Co-culturing the transplant or part thereof of step a) with the exposed tissue sample of step c); and
e) Exposing the mixture (co-culture) of step d1) to a low dose of apoptotic agents.

Step d1) of co-culturing can be performed for at least 0.5 h, 1 h, 2 h, 3 h, 4 h, 5 h or 6 h.

In one embodiment, the transplant or part thereof is a donor cell population.

In one embodiment, the transplant or part thereof is a hematopoietic donor cell population.

In one embodiment, the transplant or part thereof, is obtained from the donor. In one embodiment, the transplant or part thereof, which is obtained from the donor, is a cell transplant, preferably a hematopoietic cell transplant.

In one embodiment, the transplant or part thereof, which is obtained from the donor, is derived from stem cells, bone marrow, peripheral blood, white blood cells, leukocytes or umbilical cord blood. In a preferred embodiment the transplant or part thereof is a bone marrow transplant. In another embodiment, the transplant or part thereof of step a) which is obtained from the donor, is a hematopoietic stem cell transplant.

The terms white blood cells and leukocytes include all cells derived from these populations, i.e. derived from white blood cells or leukocytes. The term “leukocytes” can be a blood or bone marrow population enriched for mononuclear cells, enriched for lymphocytes, enriched for T lymphocytes or enriched for a desired subset of T lymphocytes.

In one embodiment, the transplant or part thereof comprises leukocyte derived cells.

In a preferred embodiment, the transplant or part thereof comprises T-cells. In another embodiment, the transplant or part thereof is depleted of at least a portion of T-cells. In another embodiment, the transplant or part thereof is depleted of T-cells.

In another embodiment, the tissue sample of step b) derived from the recipient is a blood and/or spleen sample. In a preferred embodiment, the tissue sample of step b) derived from the recipient is a blood sample. In an even more preferred embodiment, the tissue sample of step b) derived from the recipient corresponds to the recipient's peripheral blood mononuclear cells (PBMC).

In one embodiment, the cells capable of presenting antigens of step b) are antigen presenting cells (APC). In a preferred embodiment the APCs of step b) comprise monocytes, macrophages, B cells and/or dendritic cells. In a more preferred embodiment, the APCs of step b) are dendritic cells. In an especially preferred embodiment, the APCs of step b) are dendritic cells obtained by plate-passage of recipient-derived monocytes.

In one example of the present invention, dendritic cells can be obtained by plate-passage of monocytes using an extracorporeal photopheresis (ECP) derived process. Methods and devices for extracorporeal activation of monocytes and generation of dendritic cells therefrom are described in WO2014/106629 A1, WO2014/106631 A1, WO2016/001405 A1, and WO2017/005700 A1, each of which is incorporated herein by reference in its entirety. ECP describes a process in which monocytes derived from a blood sample or a fraction thereof are exposed to mechanical stress (e.g., shear forces) and plasma components (e.g., platelets) or derivatives or mimics thereof, thereby activating the monocytes to differentiate into healthy, physiologic dendritic cells which are also termed phDC herein. ECP and ECP derived processes, including the differentiation of monocytes into phDC, may be performed in a large-scale ECP device, e.g., a clinical ECP device (e.g., a THERAKOS® CELLEX® device), or in a miniaturized ECP device, e.g., a Transimmunization plate as described in WO2017/005700 A1; or in a plastic bag.

The inventors found that phDC obtained by the method described above are advantageous as compared to DC obtained by other methods such as cytokines or direct isolation from the recipient, as phDC are generated physiologically (without the need for chemicals such as cytokines) with greater reproducibility and controllability under precise in vitro laboratory conditions.

Thus, in an especially preferred embodiment, phDC of the recipient are obtained by subjecting monocytes contained in a blood sample to a shear force by passing the blood sample or fraction thereof through a flow chamber of a device. Preferably, platelets are present in the flow chamber which can be either derived from the recipient's blood sample or a fraction thereof or provided separately. Additionally or alternatively, plasma components can be present in the flow chamber which can be either derived from the recipient's blood sample or a fraction thereof or provided separately.

A monocyte of a recipient may be obtained by any suitable means, e.g., from a blood sample or a fraction thereof. The fraction of the blood sample may be, e.g., a buffy coat including white blood cells and platelets. Alternatively, the fraction of the blood sample may be an isolated peripheral blood mononuclear cell (PMBC). PMBCs may be isolated from a blood sample using, e.g., centrifugation over a Ficoll-Hypaque gradient (Isolymph, CTL Scientific). In another example, the fraction of the blood sample may be a purified or enriched monocyte preparation. Monocytes may be enriched from PBMCs using, e.g., one, two, or all three of plastic adherence; CD14 magnetic bead positive selection (e.g., from Miltenyi Biotec); and a Monocyte Isolation Kit II (Miltenyi Biotec).

Any suitable volume of blood can be used. The blood sample (e.g., the blood sample from which the fraction is derived) may be between about 1 μL and about 500 mL, e.g., between about 1 μL and about 10 mL, between about 1 μL and about 5 mL, between about 1 μL and about 1 mL, between about 1 μL and about 750 μL, between about 1 μL and about 500 μL, between about 1 μL and about 250 μL, between about 10 mL and about 450 mL, about 20 mL and about 400 mL, about 30 mL and about 350 mL, about 40 mL and about 300 mL, about 50 mL and about 200 mL, or about 50 mL and about 100 mL. In some embodiments, the blood sample or the fraction thereof or the additional blood sample or the fraction thereof is less than or equal to about 100 mL (e.g., about 50 mL to about 100 mL) In some embodiments, the ECP device is a miniaturized ECP device, e.g. a transimmunization (TI) plate. In some embodiments, the ECP device is a plastic bag. The skilled person is well aware of methods to distinguish dendritic cells including phDC from monocytes, such as e.g. by assessing gene expression.

In principle, the tissue sample of the recipient comprising cells capable of presenting antigens can be combined with the donor transplant or part thereof, while the cells within the tissue sample of the recipient are proliferating and viable. However, it is preferred that the tissue sample of the recipient is treated with at least one agent or a cell lysis-inducing method, that prevents the cells from proliferating or kills them. The at least one agent can be an anti-proliferative agent, an apoptotic agent or lethal radiation or any combination thereof. The cell lysis can be performed mechanically, physically or chemically. Preferably, the cell lysis is performed by one or more cycles of freeze-thawing.

In one embodiment, exposure of the recipient tissue sample to the at least one anti-proliferative agent, at least one apoptotic agent, lethal radiation or at least one cycle of freeze-thawing results in at least 1%, 5%, 10%, 20%, 30%, 40%, 50%, 75% or 90% of the cells not proliferating or killed. The person skilled in the art may determine how many cells are not proliferating or are killed as compared to an untreated sample. One suitable method may be the MTT assay, which is based on NAD(P)H-dependent cellular oxidoreductase enzymes capable of reducing the tetrazolium dye MTT (3-(4,5-dimethylthiazol-2-yl)-2,5-diphenyltetrazolium bromide), to its insoluble formazan, which has a purple color. The cells of the recipient tissue sample may be exposed to the at least one anti-proliferative agent, at least one apoptotic agent or lethal radiation for any time suitable to release recipient antigens, e.g. 30 s, 1 min, 2 min or more. The time needed can be determined by the skilled person as compared to an untreated sample using, e.g., methods that assess cell viability. One example would be the MTT assay.

In a preferred embodiment, the anti-proliferative agent is mitomycin C. In another preferred embodiment, the apoptotic agent is the combination of a psoralen and UVA, riboflavin-phosphate and UVA and/or aminolevulinic acid and light. In case that 5-aminolevulinic acid and light are used as apoptotic agents, the light is blue light or has a wavelength of 405 nm. Particularly preferred psoralens are 8-MOP and amotosalen. The most preferred psoralen is 8-MOP. In an especially preferred embodiment, the apoptotic agent is the combination of 8-MOP and UVA. In an even more preferred embodiment, the lethal irradiation is ultraviolet irradiation, gamma irradiation, electron irradiation or X-rays. Particularly preferred is gamma irradiation.

In one embodiment, the tissue sample of the recipient comprising cells capable of presenting antigens is exposed to at least one anti-proliferative agent, at least one apoptotic agent, lethal radiation or at least one cycle of freeze-thawing.

The following quantities are for orientation purposes. The skilled person may readily find concentrations and doses to be applied which achieve the effect of rendering at least a portion of recipient cells capable of presenting antigens apoptotic. In a preferred embodiment, the concentration of mitomycin C is 0.01 mM to 1 mM. Preferably, mitomycin C is combined with ALA (5-aminolaevulinic acid) therapy. The concentration of ALA can be 0.2 mM to 20 mM (or 0.0034% to 0.335%). The accompanying light dose can be 1 J/cm² to 40 J/cm². The light can be UVA or blue light in general. In a preferred embodiment, the concentration of riboflavin-phosphate is 1 μM to 100 μM. In a preferred embodiment, the concentration of amotosalen is 50 μM to 500 μM. The light dose accompanying the afore-mentioned riboflavin or amotosalen can be 1 J/cm² to 10 J/cm². The corresponding light can be UVA or blue light in general. In a preferred embodiment, the concentration of 8-MOP is 0.2 μM to 2.5 μM (or 43 ng/mL to 540 ng/mL). The accompanying light dose can be 0.5 J/cm²-5 J/cm². The light can be UVA or blue light in general.

In one embodiment, the recipient-derived dendritic cell population and the donor cell population are combined in step d) at a ratio of from about 1:5 to about 1:500, such as about 1:10, 1:50, 1:75, 1:100, 1:150, 1:200 or 1:400. Optionally, in step d1), the recipient-derived dendritic cell population and the donor cell population are co-cultivated, for a period of time sufficient for activation of reactive immune cells, preferably T cells within the donor cell population. A sufficient time period can for example be determined by the mixed lymphocyte reaction (MLR) assay, as described for example in DeWolf et al., 2016, Transplantation, 100(8):1639-1649. Generally, a sufficient time period to activate reactive T cells is at least 5 h, 6 h, 7 h, 8 h, 9 h, 10 h, 12 h, at least 24 h or 48 h, including at least 3, 5, 7, or 10 days.

After the combination of the recipient tissue sample and the transplant in step d), or if present, the co-culturing step d1) described above, the mixture is exposed to a low dose of at least one apoptotic agent. In a preferred embodiment, the at least one apoptotic agent is the combination of a psoralen and UVA, riboflavin-phosphate and UVA and/or aminolevulinic acid and light. Particularly preferred psoralens are 8-MOP and amotosalen. The most preferred psoralen is 8-MOP. In a most preferred embodiment, the at least one apoptotic agent is the combination of 8-MOP and UVA. Embodiments should preferably be chosen so that essentially all allo-reactive T cells are in contact with the at least one apoptotic agent. In case of 8-MOP/UVA, essentially all allo-reactive T cells should be in contact with 8-MOP and exposed to UVA light. The inventors found that the dose of the at least one apoptotic agent must be low. Thus, in case of 8-MOP and UVA the dose which is applied to the mixture must be lower than the dose usually applied in classical ECP procedures, which corresponds to a typical dose of 1 J/cm² to 3 J/cm² UVA in combination with a concentration of 8-MOP of 100 ng/mL to 300 ng/mL.

In preferred embodiments, the dose of UVA is below 1 J/cm², equal to or below 0.5 J/cm², equal to or below 0.2 J/cm² or equal to or below 0.1 J/cm². In other preferred embodiments, the dose of 8-MOP is equal to or below 300 ng/mL, 250 ng/mL, 200 ng/mL or 100 ng/mL. In a preferred embodiment, the dose of 8-MOP is 200 ng/mL and the dose of UVA is 0.1 J/cm².

Expressed in fractions, a low dose UVA corresponds to ½ of 1 J/cm², ⅕ of 1 J/cm², or 1/10 of 1 J/cm², with 1 J/cm² being considered as the typical UVA dose applied in classical ECP. In a preferred embodiment, a low dose of UVA corresponds to 1/10 of the UVA dose applied in classical ECP.

In one embodiment, the dose of UV light is normalized to the number of cells within the mixture. In principle erythrocytes in the mixture may shield the white blood cells including allo-reactive T-cells from exposure to UV light. Thus, the dose of UV light which needs to be applied is dependent on the number of erythrocytes in the mixture, or the hematocrit. In one embodiment, the number of white blood cells within the mixture is 90 to 100×10⁶ and a UV dose of 0.1 J/cm² is applied.

Without being bound to a scientific theory, it is believed that the remarkable effects of the present invention are due, at least in part, to the low dose of 8-MOP and UVA which leads to a selective reduction or elimination of allo-reactive immune cells, in particular allo-reactive donor T cells.

In one embodiment, the recipient and donor are mammalian. Mammals include for example, but are not limited to, humans, non-human primates, pigs, dogs, cats and rodents. In a preferred embodiment, the recipient and donor are human.

All of the above embodiments may be carried out in vitro.

The methods of the present invention can be applied in conjunction with other therapies for the treatment of immune defects associated with hematopoietic stem cell transplantation.

For the following embodiment of the first aspect, all of the above embodiments relating to the first aspect apply mutatis mutandis:

In one embodiment, the invention relates to a method comprising the following steps:
a) Exposing a tissue sample obtained from a future recipient to at least one anti-proliferative agent, at least one apoptotic agent, lethal radiation or at least one cycle of freeze-thawing;
b) Combining a transplant or part thereof obtained from a donor with the exposed tissue sample of step a); and
c) Exposing the combination of step b) to at least one apoptotic agent.

The methods of the invention, or specific steps of the methods, can be practiced in a bag such as a plastic bag. If plastic materials are considered, one may use bags made of plastic films based on: polyolefin, polyethylene, fluoropolymer, polyvinyl chloride, ethylene-vinyl acetate-copolymer, ethylene vinyl alcohol, polyvinylidene fluoride, or other plastic films approved for medical use. In a preferred embodiment of the present invention, the bag is made of ethylen-vinyl acetate-copolymer. The bag may be made of a material that provides a degree of transparency such that the sample or cell mixture can be irradiated with visible or UV light.

### Second Aspect: A Transplant or Part Thereof Obtained by a Method According to the First Aspect

In a second aspect, the present invention relates to a transplant or part thereof obtained by a method according to the first aspect (including all embodiments as described above).

In one embodiment, the transplant or part thereof obtained by a method according to the first aspect has reduced immunogenicity. Put in other words, the present invention provides in a second aspect a transplant or a part thereof with fewer allo-reactive immune cells, in particular fewer allo-reactive T cells. In a preferred embodiment, the transplant or a part thereof has less allo-reactive donor T cells.

In one embodiment, the immunoreactivity caused by the transplant or part thereof is reduced.

### Third Aspect: A Transplant or Part Thereof According to the Second Aspect for Use in a Method of Preventing or Reducing Graft-Versus-Host Disease

In a third aspect, the present invention relates to a transplant or part thereof according to the second aspect (including all embodiments of the first and second aspect as described above) for use in a method of preventing or reducing graft-versus-host disease.

In one embodiment, a transplant or part thereof, having reduced immunogenicity, which has been obtained by the methods of the first aspect, is for use in the treatment of an allogeneic or haploidentical recipient in need of a transplant. In another embodiment, a transplant or part thereof, having fewer allo-reactive donor T cells, which has been obtained by the methods of the first aspect, is for use in the treatment of an allogeneic or haploidentical recipient in need of a transplant. Following such a treatment, which takes place prior to transplantation, the risk of developing GvHD, and the severity of GvHD if it develops, is significantly reduced compared with administering an untreated transplant.

Further preferred embodiments of the present invention relate to:
1. Apoptotic agent treated transplant or part thereof for use in a method of preventing or reducing graft versus host disease, the method comprising the following steps:
   a) Providing a transplant or a part thereof from the donor;
   b) Providing a tissue sample from the recipient comprising cells capable of presenting antigens;
   c) Exposing the tissue sample of step b) to at least one anti-proliferative agent, at least one apoptotic agent, lethal radiation or at least one cycle of freeze-thawing;
   d) Combining the transplant or a part thereof of step a) with the exposed tissue sample of step c);
   e) Exposing the combination of step d) to a low dose of at least one apoptotic agent; and
   f) Transferring the exposed combination of step e) to the recipient.
2. Transplant or part thereof for use according to 1, wherein the method further comprises step d1) of co-culturing the transplant or part thereof of step a) with the exposed tissue sample of step c).
3. Transplant or part thereof for use according to 2, wherein step d1) of co-culturing is performed for at least 0.5 h, 1 h, 2 h, 3 h, 4 h, 5 h or 6 h.
4. Transplant or part thereof for use according to any of 1 to 3, wherein the transplant of step a) is derived from stem cells, bone marrow, peripheral blood, white blood cells, leukocytes or umbilical cord blood.
5. Transplant or part thereof for use according to any of 1 to 4, wherein the transplant or part thereof of step a) comprises T-cells.
6. Transplant or part thereof for use according to any of 1 to 5, wherein the transplant or part thereof of step a) has been depleted of at least a portion of T-cells.
7. Transplant or part thereof for use according to any of 1 to 6, wherein the transplant or part thereof of step a) is an allogeneic or haploidentical transplant.
8. Transplant or part thereof for use according to any of 1 to 7, wherein the tissue sample of step b) is derived from blood and/or spleen of the recipient.
9. Transplant or part thereof for use according to any of 1 to 8, wherein the cells capable of presenting antigens from the recipient of step b) are antigen presenting cells.
10. Transplant or part thereof for use according to any of 1 to 9, wherein the antigen presenting cells are dendritic cells.
11. Transplant or part thereof for use according to any of 1 to 10, wherein the dendritic cells have been obtained by plate passage of PBMC from the recipient.
12. Transplant or part thereof for use according to any of 1 to 11, wherein the at least one anti-proliferative agents of step c) is mitomycin C.
13. Transplant or part thereof for use according to any of 1 to 12, wherein the at least one apoptotic agent of step c) is the combination of a psoralen and UVA, riboflavin-phosphate and UVA and/or 5-aminolevulinic acid and light.
14. Transplant or part thereof for use according to 13, wherein the psoralen is 8-MOP or amotosalen.
15. Transplant or part thereof for use according to any of 1 to 14, wherein the lethal irradiation of step c) is ultraviolet radiation, gamma radiation, electron radiation or X-rays.
16. Transplant or part thereof for use according to any of 1 to 15, wherein the at least one apoptotic agent of step e) is the combination of a psoralen and UVA, riboflavin-phosphate and UVA and/or 5-aminolevulinic acid and light.
17. Transplant or part thereof for use according to 16, wherein the psoralen is 8-MOP or amotosalen.
18. Transplant or part thereof for use according to 17, wherein the psoralen is 8-MOP.
19. Transplant or part thereof for use according to 18, wherein the dose of 8-MOP is equal to or below 200 ng/mL.
20. Transplant or part thereof for use according to 16, wherein the dose of UVA is equal to or below 1 J/cm², 0.5 J/cm², 0.2 J/cm² or 0.1 J/cm².
21. Transplant or part thereof for use according to any of 1 to 20, wherein the recipient's immune system has not been treated with radiation or chemotherapy before the transplantation.
22. Transplant or part thereof for use according to any of 1 to 21, wherein the donor and recipient are mammalian, preferably human.
23. Transplant or part thereof for use according to any of 1 to 22, wherein the recipient is not treated with immunosuppressants prior to and/or after transplantation.
24. Transplant or part thereof for use according to any of 1 to 23, wherein excessive tumor growth in the recipient after transplantation is prevented or reduced.

The transplant of the present invention can be administered in conjunction with other therapies for the treatment of immune defects associated with hematopoietic stem cell transplantation.

### Fourth Aspect: Method of Reducing or Preventing Graft-Versus-Host Disease Using a Transplant or Part Thereof Obtained According to a Method of the First Aspect

In a fourth aspect, the invention provides for a method of reducing or preventing graft-versus-host disease using a transplant or part thereof obtained according to a method of the first aspect (including all embodiments as described above).

In one embodiment, a method of preventing or reducing graft-versus host disease is provided, wherein a transplant or part thereof, having reduced immunogenicity, which has been obtained by the methods of the first aspect, is administered to a subject in need thereof. In another embodiment, a method of preventing or reducing graft-versus host disease is provided, wherein a transplant or part thereof, having fewer allo-reactive donor T-cells, which has been obtained by the methods of the first aspect, is administered to a subject in need thereof. Following such an administration, which takes place prior to transplantation, the risk of developing GvHD, and the severity of GvHD if it develops, is significantly reduced compared with administering an untreated transplant.

### Fifth Aspect: A Method to Selectively Reduce Immunogenicity in a Transplant Using at Least One Recipient Antigen

The first aspect of the invention as described above relies on the stimulation of allo-reactive immune cells in the transplant, in particular allo-reactive donor T-cells, by adding cells which are derived from the recipient, preferably PBMCs and/or splenocytes. These recipient cells contain mismatched MHC molecules which activate allo-reactive immune cells, in particular allo-reactive donor T-cells. However, the stimulation of allo-reactive immune cells in the transplant, in particular allo-reactive donor T-cells and their selective deletion can principally occur based on at least one recipient antigen, e.g. a recipient-specific cell-free source of mismatched MHC such as cell-free proteins.

Utilization of a cell-free system or an antigen based system has several advantages. As the recipient's stimulatory molecules (in the following “recipient's sample”) are cell-free, the risk of contamination from unwanted cells is reduced, for example contaminating circulating malignant cells, or inadequately killed recipient cells that could react in detrimental ways against important stem cells and limit successful engraftment, or infectious agents or infected cells, etc. A cell-free recipient's sample also provides for better reliability. The approach is more streamlined and quality controlled, for example, where titratable quantities of characterized and uniform antigen (for example, mismatched MHC proteins) are given to each patient's incoming graft cells. A cell-free recipient's sample removes a source of potential variability that could hinder the treatment's breadth of effectiveness. Moreover, antigen selectivity is modular. A cell-free option or antigen based option provides control over the specific antigens to which donor cells (T cells) will be tolerized against. Clones of T cells that will be deleted can be deliberately chosen by designing e.g. a cocktail of proteins (including, but not limited to MHCs or other recipient-specific proteins) to prevent allo-reactivity against. Aside from mismatched MHC, specific peptide-MHC combinations are important antigen targets, as there is evidence for deleting allo-reactive T cells responding against peptides in mismatched MHC (Wang et al., How an alloreactive T-cell receptor achieves peptide and MHC specificity. Proc. Natl. Acad. Sci. U.S.A 114, E4792-E4801 (2017)). Inclusion of non-MHC antigen are also important as protein targets as they have been shown to be also responsible for alloreactivity in transplants (see Melenhorst et al. Alloreactivity Across HLA Barriers Is Mediated by Both Naïve and Antigen-Experienced T Cells. Biol. Blood Marrow Transplant. 17, 800-809 (2011)). Finally, cell-free recipient's sample obviates additional recipient cell preparation prior to transplant, which may include freezing down recipient cells, thawing them, gamma/PUVA irradiating them prior to addition to a graft.

Therefore, in a fifth aspect, the invention relates to a method comprising the following steps:
a) Providing a transplant or a part thereof from a donor;
b) Providing at least one recipient antigen;
c) Combining the transplant or part thereof of step a) with the at least one recipient antigen of step b); and
d) Exposing the combination of step c) to a low dose of at least one apoptotic agent.

In one embodiment, the at least one recipient antigen is a cell-free recipient sample.

In one embodiment, the method is performed prior to transplantation.

In one embodiment the method is for selectively reducing immunogenicity in a transplant or a part thereof prior to transplantation.

In one embodiment, the method is for selectively reducing or eliminating allo-reactive immune cells, in particular allo-reactive donor T-cells from the transplant prior to transplantation.

In one embodiment, the method is for selectively reducing immunogenicity in a transplant or a part thereof prior to transplantation and selectively reducing or eliminating reactive donor T cells from the transplant prior to transplantation.

In one embodiment of the fifth aspect, the invention relates to a method comprising the following steps:
a) Providing a transplant or a part thereof from a donor;
b) Providing at least one recipient antigen;
c) Combining the transplant or part thereof of step a) with the at least one recipient antigen of step b);
c1) Co-culturing the transplant or part thereof of step a) with the at least one recipient antigen of step b); and
d) Exposing the mixture (co-culture) of step c1) to a low dose of apoptotic agents.

Step c1) of co-culturing can be performed for at least 0.5 h, 1 h, 2 h, 3 h, 4 h, 5 h or 6 h.

In one embodiment, the transplant or part thereof is a donor cell population.

In one embodiment, the transplant or part thereof is a hematopoietic donor cell population. In one embodiment, the transplant or part thereof, is obtained from the donor. In one embodiment, the transplant or part thereof, which is obtained from the donor, is a cell transplant, preferably a hematopoietic cell transplant.

In one embodiment, the transplant or part thereof, which is obtained from the donor, is derived from stem cells, bone marrow, peripheral blood, white blood cells, leukocytes or umbilical cord blood. In a preferred embodiment the transplant or part thereof is a bone marrow transplant.

In another embodiment, the transplant or part thereof of step a) which is obtained from the donor, is a hematopoietic stem cell transplant.

The terms white blood cells and leukocytes include all cells derived from these populations, i.e. derived from white blood cells or leukocytes. The term “leukocytes” can be a blood or bone marrow population enriched for mononuclear cells, enriched for lymphocytes, enriched for T lymphocytes or enriched for a desired subset of T lymphocytes.

In one embodiment, the transplant or part thereof comprises leukocyte derived cells.

In a preferred embodiment, the transplant or part thereof comprises T-cells. In another embodiment, the transplant or part thereof is depleted of at least a portion of T-cells. In another embodiment, the transplant or part thereof is depleted of T-cells.

In one embodiment, the at least one recipient antigen of step b) is an antigen derived from a protein, peptide, MHC molecule or fragment thereof, peptide-MHC combination, cell lysate or any combination thereof. Thus, the antigen is part of an antigenic molecule which is a protein, peptide, MHC molecule or fragment thereof, peptide-MHC combination, cell lysate or any combination thereof.

In one embodiment, the at least one recipient antigen of step b) is derived from a protein or, put in other words, corresponds to a part of an antigenic molecule which is a protein. The protein can be unmodified or recombinant, or a combination thereof. The protein can be provided as mRNA which is translated in a cellular or cell-free translation system. In one embodiment the protein is cell-free.

In one embodiment, the at least one recipient antigen of step b) is a peptide, is derived from a peptide, or, corresponds to a part of an antigenic molecule which is a peptide. The peptide can be unmodified or recombinant. The peptides can be provided as mRNA which is translated in a cellular or cell-free translation system. In one embodiment the peptides are cell-free.

In one embodiment, the at least one recipient antigen of step b) comprises antigens from a cell-free sample.

In one embodiment, the at least one recipient antigen of step b) is a MHC molecule, is derived from a MHC molecule, or, corresponds to a fragment of a MHC molecule.

In one embodiment, the at least one recipient antigen of step b) is an antigen from cell-free MHC molecules or fragments thereof. In one embodiment, the at least one recipient antigen of step b) is an antigen from mismatched MHC molecules or fragments thereof.

In one embodiment, the at least one recipient antigen of step b) is an antigen from peptide-MHC combinations. In one embodiment, the at least one recipient antigen of step b) corresponds to non-MHC antigens. In one embodiment, the at least one recipient antigen of step b) corresponds to a cell-lysate.

Sources for MHC molecules include but are not limited to recombinant and cell-derived sources. The MHC molecules can be monomeric or oligomeric MHC molecules, i.e. monomers, tetramers, multimers etc. In one embodiment, the MHC molecules or fragments thereof are derived from platelets; cell lysates; exosomes, nanoparticles or similar polymeric spheres or scaffolds; artificial or genetically engineered cells or platelets; or, virions or viral like particles expressing or containing MHC.

In case of exosomes, nanoparticles or similar polymeric sphere or scaffold which contain MHC, the exosomes, nanoparticles or similar polymeric spheres or scaffolds are coated with stochiometric amounts of the same or different MHC molecules.

In case of artificial or genetically engineered cells or platelets, for example, a universal human donor cell line could be created by CRISPR-cas9 technology which would express a large panel of surface MHC molecules which could be fixed by safe non-chemical means, cryopreserved and then added fresh to graft by methodologies similar to acellular reagents above.

In another embodiment, the MHC molecules or fragments thereof are derived from mRNA which is translated in a cellular or cell-free translation system. The use of cell-free MHC proteins has been previously demonstrated, and is a widely-accepted methodology. MHC molecules and peptide-MHCs have been extensively utilized for research into TCR-MHC interactions (see Garcia et al., Structural basis of plasticity in T cell receptor recognition of a self peptide-MHC antigen. Science 279, 1166-1172 (1998); Tian et al., J. A. CD8+ T Cell Activation Is Governed by TCR-Peptide/MHC Affinity, Not Dissociation Rate. J. Immunol. 179, 2952-2960 (2007); Cai et al. Full control of ligand positioning reveals spatial thresholds for T cell receptor triggering. Nat. Nanotechnol. 1-33 (2018)), antigen-specificity (Joglekar, T cell antigen discovery. Nat. Methods (2020)) and allo-reactivity (Weng, X. et al. Allo-restricted CTLs generated by coculturing of PBLs and autologous monocytes loaded with allogeneic peptide/HLA/IgG1-Fc fusion protein. J. Leukoc. Biol. 85, 574-581 (2009); Pittet et al., Ex Vivo Characterization of Allo-MHC-Restricted T Cells Specific for a Single MHC-Peptide Complex. J. Immunol. 176, 2330-2336 (2006); Colf et al., How a Single T Cell Receptor Recognizes Both Self and Foreign MHC. Cell 129, 135-146 (2007)), as well as nanomedicine (Umeshappa et al., Suppression of a broad spectrum of liver autoimmune pathologies by single peptide-MHC-based nanomedicines. Nat. Commun. 10, 1-17 (2019)).

Optionally, in step c1), the at least one recipient antigen and the donor transplant or part thereof are co-cultivated, for a period of time sufficient for activation of reactive T cells within the donor cell population. Generally, a sufficient time period to activate reactive T cells is at least 5 h, 6 h, 7 h, 8 h, 9 h, 10 h, 12 h, at least 24 h or 48 h, including at least 3, 5, 7, or 10 days.

After the combination of the at least one recipient antigen and the transplant in step c), or if present, the co-culturing step c1) described above, the mixture is exposed to a low dose of at least one apoptotic agent. All embodiments and explanations relating to the at least one apoptotic agent as set forth in relation to the first aspect equally apply to the fifth aspect and the embodiments of the fifth aspect.

In one embodiment, the recipient and donor are mammalian. Mammals include for example, but are not limited to, humans, non-human primates, pigs, dogs, cats and rodents. In a preferred embodiment, the recipient and donor are human.

All of the above embodiments may be carried out in vitro.

The methods of the present invention can be applied in conjunction with other therapies for the treatment of immune defects associated with hematopoietic stem cell transplantation.

For the following embodiment of the fifth aspect, all of the above embodiments relating to the fifth aspect apply mutatis mutandis:

In one embodiment, the invention relates to a method comprising the following steps:
a) Combining at least one recipient antigen with a transplant or part thereof obtained from a donor; and
b) Exposing the combination of step a) to at least one apoptotic agent.

All steps above are carried out in vitro. All embodiments and explanations relating to the at least one apoptotic agent as set forth in relation to the first aspect equally apply.

The methods of the sixth aspect, or specific steps of the methods, can be practiced in a bag such as a plastic bag. If plastic materials are considered, one may use bags made of plastic films based on: polyolefin, polyethylene, fluoropolymer, polyvinyl chloride, ethylene-vinyl acetate-copolymer, ethylene vinyl alcohol, polyvinylidene fluoride, or other plastic films approved for medical use. In a preferred embodiment of the present invention, the bag is made of ethylen-vinyl acetate-copolymer. The bag may be made of a material that provides a degree of transparency such that the sample or cell mixture can be irradiated with visible or UV light.

### Sixth Aspect: A Transplant or Part Thereof Obtained by a Method According to the Fifth Aspect

In a sixth aspect, the present invention relates to a transplant or part thereof obtained by a method according to the fifth aspect (including all embodiments as described above).

In one embodiment, the transplant or part thereof obtained by a method according to the fifth aspect has reduced immunogenicity. Put in other words, the present invention provides in a fifth aspect a transplant or a part thereof with less allo-reactive immune cells, in particular less allo-reactive T-cells. In a preferred embodiment, the transplant or a part thereof has less allo-reactive donor T-cells.

### Seventh Aspect: A Transplant or Part Thereof According to the Sixth Aspect for Use in a Method of Preventing or Reducing Graft-Versus-Host Disease

In a seventh aspect, the present invention relates to a transplant or part thereof according to the sixth aspect (including all embodiments of the fifth and sixth aspect as described above) for use in a method of preventing or reducing graft-versus-host disease.

In one embodiment, a transplant or part thereof, having reduced immunogenicity, which has been obtained by the methods of the fifth aspect, is for use in the treatment of an allogeneic or haploidentical recipient in need of a transplant. In another embodiment, a transplant or part thereof, having less alloreactive donor T cells, which has been obtained by the methods of the fifth aspect, is for use in the treatment of an allogeneic or haploidentical recipient in need of a transplant. Following such a treatment, which takes place prior to transplantation, the risk of developing GvHD, and the severity of GvHD if it develops, is significantly reduced compared with administering an untreated transplant.

Further preferred embodiments of the present invention relate to:
1. Apoptotic agent treated transplant or part thereof for use in a method of preventing or reducing graft versus host disease, the method comprising the following steps:
   a) Providing a transplant or a part thereof from a donor;
   b) Providing at least one recipient antigen;
   c) Combining the transplant or a part thereof of step a) with the at least one recipient antigen of step b);
   d) Exposing the combination of step c) to a low dose of at least one apoptotic agent; and
   e) Transferring the exposed combination of step d) to the recipient.
2. Transplant or part thereof for use according to 1, wherein the method further comprises step c1) of co-culturing the transplant or part thereof of step a) with the at least one recipient antigen of step b).
3. Transplant or part thereof for use according to 2, wherein step c1) of co-culturing is performed for at least 0.5 h, 1 h, 2 h, 3 h, 4 h, 5 h or 6 h.
4. Transplant or part thereof for use according to any of 1 to 3, wherein the transplant of step a) is derived from stem cells, bone marrow, peripheral blood, white blood cells, leukocytes or umbilical cord blood.
5. Transplant or part thereof for use according to any of 1 to 4, wherein the transplant or part thereof of step a) comprises T-cells.
6. Transplant or part thereof for use according to any of 1 to 4, wherein the transplant or part thereof of step a) has been depleted of at least a portion of T-cells.
7. Transplant or part thereof for use according to any of 1 to 6, wherein the transplant or part thereof of step a) is an allogeneic or haploidentical transplant.
8. Transplant or part thereof for use according to any of 1 to 7, wherein the at least one recipient antigen of step b) is a protein, peptide, MHC molecule or fragment thereof, peptide-MHC combination, cell lysate or any combination thereof.
9. Transplant or part thereof for use according to any of 1 to 8, wherein the at least one apoptotic agent of step e) is the combination of a psoralen and UVA, riboflavin-phosphate and UVA and/or 5-aminolevulinic acid and light.
10. Transplant or part thereof for use according to 9, wherein the psoralen is 8-MOP or amotosalen.
11. Transplant or part thereof for use according to 9 or 10, wherein the psoralen is 8-MOP.
12. Transplant or part thereof for use according to 11, wherein the dose of 8-MOP is equal to or below 200 ng/mL.
13. Transplant or part thereof for use according to 9, wherein the dose of UVA is equal to or below 1 J/cm², 0.5 J/cm², 0.2 J/cm² or 0.1 J/cm².
14. Transplant or part thereof for use according to any of 1 to 13, wherein the recipient's immune system has not been treated with radiation or chemotherapy before the transplantation.
15. Transplant or part thereof for use according to any of 1 to 14, wherein the donor and recipient are mammalian, preferably human.
16. Transplant or part thereof for use according to any of 1 to 15, wherein the recipient is not treated with immunosuppressants prior to and/or after transplantation.
17. Transplant or part thereof for use according to any of 1 to 16, wherein excessive tumor growth in the recipient after transplantation is prevented or reduced.

The transplant of the present invention can be administered in conjunction with other therapies for the treatment of immune defects associated with hematopoietic stem cell transplantation.

### Eighth Aspect: Method of Reducing or Preventing Graft-Versus-Host Disease Using a Transplant or Part Thereof Obtained According to a Method of the Fifth Aspect

In an eighth aspect, the invention provides for a method of reducing or preventing graft-versus-host disease using a transplant or part thereof obtained according to a method of the fifth aspect (including all embodiments as described above).

In one embodiment, a method of preventing or reducing graft-versus host disease is provided, wherein a transplant or part thereof, having reduced immunogenicity, which has been obtained by the methods of the fifth aspect, is administered to a subject in need thereof. In another embodiment, a method of preventing or reducing graft-versus host disease is provided, wherein a transplant or part thereof, having less allo-reactive donor T-cells, which has been obtained by the methods of the fifth aspect, is administered to a subject in need thereof. Following such an administration, which takes place prior to transplantation, the risk of developing GvHD, and the severity of GvHD if it develops, is significantly reduced compared with administering an untreated transplant.

Ninth Aspect: Compositions that Include a Transplant from a Donor, at Least One Recipient Antigen and an Apoptotic Agent or an Anti-Proliferative Agent

In a ninth aspect, the invention relates to a composition that includes (a) a transplant from a donor, or a part thereof; (b) at least one recipient antigen; and (c1) an apoptotic agent or (c2) an anti-proliferative agent.

The composition may include any transplant or part thereof, including any transplant or part thereof disclosed in the aspects mentioned above.

In some embodiments, the transplant from a donor, or part thereof, has a reduced immunogenicity, e.g., compared to a transplant from a donor or a part thereof that has not been exposed to at least one recipient antigen and an apoptotic agent or anti-proliferative agent.

In some embodiments, the composition includes at least one recipient antigen. The at least one recipient antigen may correspond to a tissue sample from a recipient as defined in the first aspect. Thus, the at least one recipient antigen may correspond to a blood and/or spleen sample obtained from the recipient. In some embodiments, the at least one recipient antigen corresponds to cells capable of presenting antigens. In some embodiments, the at least one recipient antigen corresponds to dendritic cells obtained from the recipient. In some embodiments, the dendritic cells have been obtained by plate-passage of PBMCs obtained from the recipient.

In some embodiments, the composition includes at least one recipient antigen as defined in the fifth aspect. Thus, the at least one recipient antigen may correspond to a protein, peptide, MHC molecule or fragment thereof, peptide-MHC combination, cell lysate or any combination thereof.

In some embodiments, the composition includes one type of recipient antigen. In other embodiments, the composition may include two, three, four, five, ten, or more different recipient antigens.

The composition may include any suitable apoptotic agent, including any apoptotic agent disclosed in the aspects mentioned above. In some embodiments, the apoptotic agent is a psoralen. In some embodiments, the psoralen is 8-MOP. In some embodiments, the composition contains at least one psoralen/UVA DNA adduct. In some embodiments, the composition contains 5%, 10%, 15%, 20% or more psoralen/UVA DNA adducts. The percentage of psoralen/UVA DNA adducts can be determined as compared to a composition without a psoralen. For example, psoralen/UVA DNA adducts can be identified using specific antibodies.

The composition may include any suitable anti-proliferative agent, including any anti-proliferative agent disclosed in the aspects mentioned above. In some embodiments, the apoptotic agent is mitomycin C.

In some embodiments, the composition contains a transplant with reduced proliferative capacity of reactive donor T cells. In some embodiments, the proliferative capacity of reactive donor T cells is at least 10%, 20%, 30%, 40%, 50%, 60%, 70%, 80%, 90%, 95%, 98% reduced or more. The reduction can be determined as compared to a sample of the same composition without the apoptotic agent or anti-proliferative agent. In some embodiments, the composition contains a transplant with at least a fraction of apoptotic cells. In some embodiments, the fraction of apoptotic cells is at least 10%, 20%, 30%, 40%, 50% or 60%, or more. In some embodiments, the fraction of apoptotic cells is at least 10%, 20%, 30%, 40%, 50% or 60%, or more of allo-reactive donor T-cells.

Further embodiments will be described hereinafter.

### FIGURE LEGENDS

FIG. 1 Schematic design of Balb/C→B6 full mismatch GVHD system; results are also depicted.

FIG. 2 Ex vivo psoralen UVA treatment (PUVA) of graft ameliorates GVHD in a full MHC mismatch model. Mice were injected s.c. with 2×10⁵ MC38 tumor cells before transplant on day −4 or the day of transplant on day 0. Mice were lethally irradiated at 950 cGy on day −1. On day 0, they underwent Balb/c→B6 transplant. They received i.v. injections of 5×10⁶ allogeneic T cell depleted bone marrow (BM) cells along with 10×10⁶ splenocytes unmanipulated, or following ex vivo PUVA treatment of the allo-stimulated graft. As controls, a group of mice received syngeneic BM and splenocytes following tumor inoculation. (A, panel 1-3) Pooled data of average weight, GvHD score and survival of all groups. (B) Average tumor volume.

FIG. 3 Ex vivo PUVA mitigates GVHD to levels comparable to PTCy. Transplant backbone: Balb/c→B6 following 950 cGy conditioning. Prior to injection, Balb/c graft was stimulated with killed B6 splenocytes for 4-5 hrs, and subsequently pulsed with titrating doses of PUVA normalized to 100×10⁶ total cells.

### DETAILED DESCRIPTION OF THE INVENTION

The present invention is based to some extent on data presented hereinafter, which showed that reactive donor T-cells in a donor transplant can be selectively reduced or eliminated.

In detail, the inventors found that alloreactive or antigen-reactive T cells from a hematopoietic donor cell population can be reduced, by co-culturing a recipient-derived stimulatory cell population with the donor cell population, under conditions wherein the recipient-derived population activates donor T cells in the donor cell population. The inventors found that the recipient-derived stimulatory cell population can correspond to at least one recipient antigen. Moreover it was found that phDC obtained by plate-passage of monocytes of the recipient activate allo-reactive T cells very efficiently, allowing for an effective reduction or elimination of these cells from a transplant or donor cell population.

During activation, the allo-reactive T-cells are particularly vulnerable to low doses of apoptotic agents such as psoralens and UVA (PUVA), in particular the combination of 8-MOP/UVA, which leads to the selective reduction or removal of the activated T cells. Donor cell populations (in other words a graft, transplant or part thereof) treated as described above can be used in reducing or preventing inflammatory conditions and in transplant procedures, such as to reduce the risk of developing graft-versus host disease. In addition, a graft-versus-leukemic cell reaction or graft-versus tumor reaction can be retained or induced.

In one embodiment, recipients who have an infection caused by viruses and/or bacteria are excluded.

The inventors found that although reactive donor T cells are reduced or eliminated, it appears that donor T cells which are reactive against possible tumors in the recipient are not reduced or eliminated to a similar extent as the reactive T cells which cause graft-versus-host disease. Thus, a graft versus tumor effect can still be maintained or induced in the transplant treated according to the above.

The inventors found that the same appears to be true for opportunistic viruses. Irradiation of recipient tissue may also lead to release of virus antigens to which reactive donor T cells may be activated. However, the inventors found that a significant reduction in the reaction to opportunistic viruses in the recipient such as JC polyoma virus is not expected. It is hypothesized that this is due to the large quantitative difference in virus antigens (and also tumor antigens) and MHC I and II antigens in the tissue sample of the future recipient.

Among the advantages of the methods of the present invention is that the use of globally immunosuppressive drugs in transplant recipients may be reduced or eliminated, thereby reducing or eliminating the adverse health effects associated with immunosuppressive drugs such as cyclophosphamide. Another advantage of the methods of the present invention is that the incidence and severity of GvHD in bone marrow/stem cell transplant recipients may be greatly reduced. A further advantage of the methods of the present invention is that the pool of potential transplant donors may be expanded. Other advantages of the methods of the present invention will be readily apparent to those skilled in the art based on the summary of the invention and preferred embodiments as set forth above.

The following general definitions are provided.

Where the term “comprising” is used in the present description and claims, it does not exclude other elements. If hereinafter a group is defined to comprise at least a certain number of embodiments, this is also to be understood to disclose a group, which preferably consists only of these embodiments or essentially only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody, which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of 20%, preferably +15%, more preferably +10%, and even more preferably +5%.

Furthermore, the terms “first”, “second”, “third” or “(a)”, “(b)”, “(c)”, “(d)” or “(i)”, “(ii)”, “(iii)”, “(iv)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” or “(i)”, “(ii)”, “(iii)”, “(iv)” etc. relate to steps of a method or use or assay there is no time or time interval coherence between the steps unless indicated otherwise, i.e. the steps may be carried out simultaneously or there may be time intervals of seconds, minutes, hours, days, weeks, months or even years between such steps, unless otherwise indicated in the application as set forth herein above or below.

Technical terms are used by their common sense. If a specific meaning is conveyed to certain terms, definitions of terms will be given in the following in the context of which the terms are used.

As used herein, “transplant” refers to any sample of cells that is removed from a mammalian individual (a “donor”) and is suitable to be reintroduced, in whole or in part, into the same (“autologous”) or different (“allogeneic”) mammalian individual (a “recipient”). The transplant can be either freshly obtained, cultured or frozen, but has been maintained under conditions suitable to maintain sterility and promote viability. A transplant contains donor T cells, some of which are anti-recipient tissue antigen donor T cells.

As used herein, the term “hematopoietic donor cell population” refers to any population of cells derived from a hematopoietic tissue that is removed from a donor and is suitable to be reintroduced, in whole or in part, into the same or different recipient.

The methods of the invention are practiced by co-culturing the recipient-derived stimulatory cell population with a transplant so as to activate donor T cells, and killing or removing the activated T cells, thereby reducing or eliminating reactive T cells from the transplant. As used herein, the term “reactive T cell” refers to a T cell present in a donor transplant that has the potential to recognize, become activated and proliferate in response to an alloantigen (producing an “allo-reactive T cell”), or other antigen presented by the recipient-derived stimulatory cell population. In one embodiment, the term “reactive T cell” refers to a T cell present in a donor transplant that shows reactivity which is restricted to non-self HLA molecules. Put in other words, in one embodiment, the T cells are allo-HLA-reactive T cells.

As used herein, the term “antigen” refers to a cell, compound, molecule, composition, or substance that can stimulate the production of antibodies or a T cell response in an animal, including compositions that are injected or absorbed into an animal. An antigen or antigenic molecule reacts with the products of specific humoral or cellular immunity, including those induced by heterologous immunogens. The term is used interchangeably with the term “immunogen”. The term “antigen” or “antigenic molecule” includes all related antigenic epitopes. An “antigenic polypeptide” is a polypeptide to which an immune response, such as a T cell response or an antibody response, can be stimulated. “Epitope” or “antigenic determinant” refers to a site on an antigen to which B and/or T cells respond. Epitopes can be formed both from contiguous amino acids (linear) or noncontiguous amino acids juxtaposed by tertiary folding of an antigenic polypeptide (conformational). Epitopes formed from contiguous amino acids are typically retained on exposure to denaturing solvents whereas epitopes formed by tertiary folding are typically lost on treatment with denaturing solvents. Normally, a B-cell epitope will include at least about 5 amino acids but can be as small as 3-4 amino acids. A T-cell epitope, such as a CTL epitope, will include 25 at least about 7-9 amino acids, and a helper T-cell epitope at least about 12-20 amino acids. Normally, an epitope will include between about 5 and 15 amino acids, such as, 9, 10, 12 or 15 amino acids. The amino acids are in a unique spatial conformation. In one example, the recipient antigen includes antigens from lymphocytes, leukocytes, such as peripheral blood leukocytes (including monocytes or monocyte-derived cells, such as dendritic cells) or a combination thereof. In some examples, recipient antigen includes lysed cell membranes from recipient peripheral blood leukocytes, spleen cells or bone marrow cells.

As used herein, the term “immunogenicity” refers to the ability of a substance, a cell or a part thereof, such as an antigen, to provoke an immune response in the body of a human or animal. Reduced immunogenicity can be determined by comparing the transplant treated with a method according to the invention to a transplant or a part thereof from the same source that has not been treated (e.g. no exposure to recipient antigen presenting cells and/or a low dose of an apoptotic agent). An assay suitable for determining reduced immunogenicity may inter alia be a MLR assay.

As used herein, the term “cell(s) capable of presenting antigens” or similar expressions refers to a cell or cells which are in principle able to present antigens on their cell surface. Examples of cells capable of presenting antigens are antigen presenting cells (APC), diseased cells such as a virus-infected cells or malignant cells. Cells present antigens in the context of MHC molecules (MHC I and MHCII), in particular MHC Class I molecules. APCs are cells that are capable of activating T cells, and include, but are not limited to, monocytes, monocyte-derived cells, macrophages, B cells and dendritic cells. In the context of the present invention APCs are also referred to as stimulatory cells or a stimulatory cell population which are used synonymously. It is to be understood that stimulatory cells are derived from the recipient.

In allograft procedures, donor T cells are reactive with recipient alloantigens. As used herein, the term “allo-antigen” refers to class I and class II major histocompatibility (MHC) or HLA antigens, as well as minor histocompatibility antigens, that differ between individuals, and which are naturally present on the surface of cells in the recipient-derived stimulatory cell population. The methods of the invention can be practiced with individuals who are closely HLA-matched, sharing all or nearly all of their class I and class II HLA antigens; haploidentical, such as siblings sharing half of their HLA antigens; or unrelated, and thus poorly HLA matched. The degree of HLA identity between individuals can readily be demonstrated by methods known in the art, including the polymerase chain reaction, mixed lymphocyte reactions (MLR), and serological measurements. In allograft procedures, the methods of the invention can thus be used to activate and reduce or eliminate donor T cells with the potential to react with alloantigens present on the surface of recipient-derived stimulatory cells, so as to reduce the risk of the recipient developing graft-versus-host disease.

As used herein, the term “reducing” refers to any method of treating the transplant or part thereof such that it contains fewer reactive donor T cells after treatment than before treatment or such that the proliferative capacity of reactive donor T-cells is reduced. Preferably, the reducing method is efficient, such that the proliferative capacity of reactive donor T cells is at least 20%, 30%, 40%, 50%, 60%, 70%, 80%, 90%, 95%, 98% reduced or more. The term “reducing” includes eliminating which implies an efficiency of reducing reactive T cells by 100%.

As used herein, the term “animal” refers to living multi-cellular vertebrate organisms, a category that includes, for example, mammals and birds. The term mammal includes both human and non-human mammals. Similarly, the term “subject” includes both human and veterinary subjects. Preferably, the mammal of the present invention is a human subject.

As used herein, the term “exposing” as used herein, refers to bringing into the state or condition of immediate proximity or direct contact.

As used herein, the term “proliferation” as used herein, means to grow or multiply by producing new cells.

The invention is now described with respect to some specific examples which, however, are for illustrative purposes and not to be construed in a limiting manner.

### EXAMPLES

### Experiment 1—Prevention of GvHD by PUVA Treated Transplant

### Materials and Methods

1 On day −4, the future graft recipient mice (C57BL/6, H2b MHC haplotype) were inoculated subcutaneously (flank) with a C57BL/6 tumor (MC38 colon carcinoma).
2 On day −1, the future graft recipient (tumor-bearing) received a lethal dose (950 cGy) of gamma irradiation that ablates his own immune system.
3 On the day of transplant (day 0), tissues were prepared for transplant.
   a. In a no-graft control, the irradiated C57BL/6 mouse did not receive any transplant.
   b. In a syngeneic control, the native C57BL/6 immune system was reconstituted by giving back C57BL/6 bone marrow and splenocytes.
   c. In an allogeneic control graft, the recipient was reconstituted with fully mismatched bone marrow and splenocytes from a Balb/c donor mouse (H2d MHC haplotype).
   d. In an allogeneic PUVA graft, Balb/c bone marrow and splenocytes were incubated for 5 hrs with lethally irradiated C57BL/6 splenocytes to activate any allo-reactive Balb/c cells in the graft, and then treated in a Petri dish with a very low dose of PUVA (200 ng/mL 8-MOP, 0.1 J/cm² UVA) to inactivate these allo-reactive Balb/c cells.
4 The prepared tissues were transplanted into the irradiated tumor-bearing recipient.
5 The recipient was then monitored for bone marrow engraftment (survival, later confirmed by blood analysis), GvHD, and tumor growth.
   The mice that were irradiated but did not receive any graft uniformly die by day 14.
   The syngeneic control mice engrafted well, did not develop GvHD (expected, as the graft is a full match), and grew large tumors.
   The allogeneic control mice engrafted well, developed severe GvHD that was uniformly lethal ˜day 25 (expected, as graft is a full mismatch), and tumor growth was difficult to assess since it is relatively slow, and lethality is fast.
   The allogeneic PUVA mice engrafted well, did not show any signs of GvHD (day 47), and although tumors grew, the growth rate appeared to be at −50% of syngeneic control mice, i.e. it was partially controlled by GvT effect.

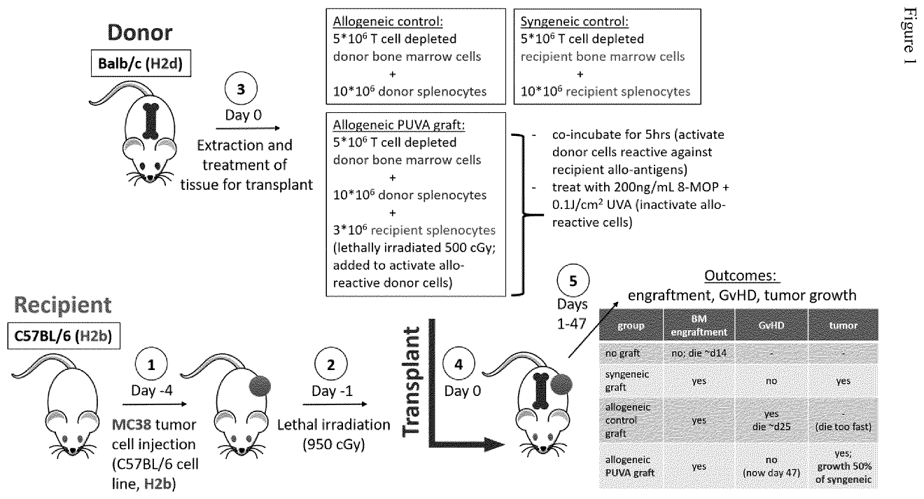

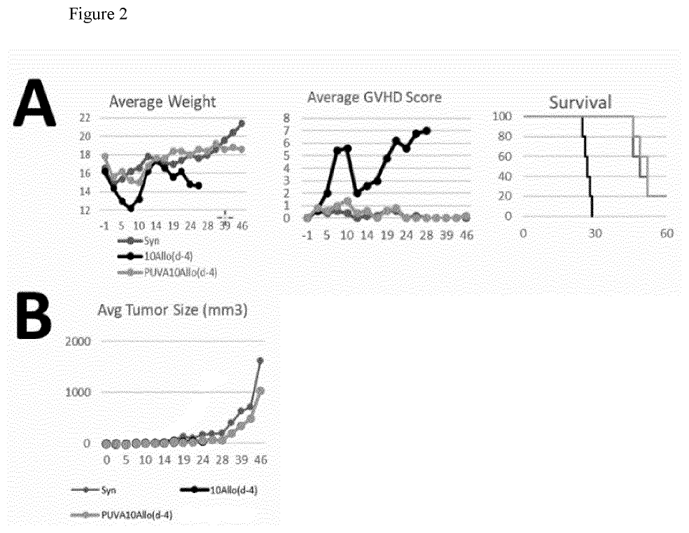

The results are depicted in Table 1 below as well as FIGS. 1 and 2. FIG. 1 also schematically depicts the above described method.

### Results

TABLE 1
Results of different treatment groups

| | Bone | | |
|---|---|---|---|
| | marrow | | |
| Group | engraftment | GvHD | Tumor |
| no graft | no; die~d 14 | — | — |
| syngeneic | yes | no | yes |
| graft | | | |
| allogeneic | yes | yes | — |
| control graft | | die~d 25 | (die too fast) |
| allogeneic | yes | no | yes; |
| PUVA graft | | (day 47) | growth 50% |
| | | | of syngeneic |

The inventors surprisingly found that a transplant treated with a low dose of 8-MOP/UVA, i.e. according to the method of the invention, prevents GvHD.

### Experiment 2—UVA Dose Dependence of Prevention of GvHD by PUVA Treated Transplant

### Materials and Methods

1 On day −1, the future graft recipient (C57BL/6 mouse, H2b MHC haplotype) received a lethal dose of gamma irradiation that ablates his own immune system.
2 On the day of transplant (day 0), tissues were prepared for transplant.
   a. In an allogeneic control graft, the recipient was reconstituted with fully mismatched bone marrow and splenocytes from a Balb/c donor mouse (H2d MHC haplotype).
   b. In an allogeneic PUVA graft experimental animals, Balb/c bone marrow and splenocytes were incubated for 5 hrs with lethally irradiated C57BL/6 splenocytes to activate any allo-reactive Balb/c T cells in the graft, and then treated in a Petri dish with a range of PUVA doses (200 ng/mL 8-MOP, and 0.05, 0.1, 0.3, or 0.6 J/cm² UVA) to inactivate these allo-reactive Balb/c T cells. Cell numbers were chosen as described in Experiment 1.
3 The prepared tissues were transplanted into the irradiated tumor-bearing recipient.
4 The recipient was then monitored for bone marrow engraftment (survival of graft recipient past 14 days, later confirmed by blood analysis), and signs of GvHD.

### Results

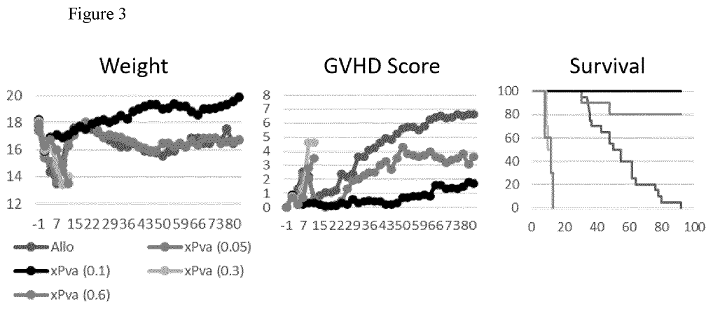

The experiment is reported in FIG. 3 at day 96, with regards to weight loss, overall GvHD score, and animal survival. N=10 mice per group.
The allogeneic control mice engrafted well, and developed severe GvHD that is uniformly lethal ˜day 25 (expected, as graft is a full mismatch).
The allogeneic PUVA mice responded to the treatment in a dose-dependent manner.
   0.05 J/cm² UVA group—mice survived but showed signs of GvHD, indicating this dose it too low to fully inactivate allo-reactive T cells.
   0.1 J/cm² UVA group—mice survived and showed very little signs of GvHD for up to 3 months post transplant, indicating this dose is very successful at inactivating allo-reactive T cells.
   0.3 J/cm² UVA group—mice did not survive past day 14 of transplant, indicating this dose damaged hematopoietic stem cells and prevented engraftment and is therefore too high.
   0.6 J/cm² UVA group—mice did not survive past day 14 of transplant, indicating this dose damaged hematopoietic stem cells and prevented engraftment and is therefore too high.

### Experiment 3—Generation of Physiologic DC

All studies were performed with blood donated by healthy human volunteers. Peripheral blood was collected into 1:100 5,000 U/mL heparin (McKesson Packaging Services), and platelet-containing PBMC isolated by density gradient centrifugation over Isolymph (CTL Scientific Supply Corp.) following the manufacturer's protocol. Autologous plasma (also containing platelets) was collected and reserved. Washed PBMC and platelets were resuspended in autologous plasma, and incubated for 1 hr either in the Transimmunziation (TI) chamber or clinical ECP plate.

In the TI chamber, the cells were passed through using a syringe pump, at a rate of 0.09 mL/min. Following plate passage, cells were collected, and the TI chamber washed with 100% FBS at 0.49 mL/min while being physically perturbed by flicking or tapping the plate surface to help detach any adherent cells from the chamber.

In the clinical ECP plate, cells were passed at a flow rate of 24 mL/min, followed by a 100 mL/min wash with human AB serum (Lonza BioWhittaker) with physical perturbation by flicking or tapping the plate surface, to help detach any adherent cells. PBMC passed through either the TI chamber or the ECP plate were collected, washed, and cultured overnight under standard conditions in RPMI without phenol-red (Gibco, Carlsbad, CA) supplemented with 15% Human AB serum (Lonza BioWhittaker), 1% penicillin/streptomycin (Invitrogen, Carlsbad, CA), and 1% L-glutamine (Invitrogen, Carlsbad, CA). The following day, physiological dendritic cells were harvested (including harvest of any attached cells by scraping).

### Experiment 4—Reducing Allo-Reactive T Cells Using Proteins, Peptides or Exosomes as Stimulator Molecules

### Materials and Methods

1 On day −1, the future graft recipient (C57BL/6 mouse, H2b MHC haplotype) receives a lethal dose of gamma irradiation that ablates his own immune system.
2 On the day of transplant (day 0), tissues are prepared for transplant.
   a. In an allogeneic control graft, the recipient is reconstituted with fully mismatched bone marrow and splenocytes from a Balb/c donor mouse (H2d MHC haplotype).
   b. In an allogeneic PUVA graft experimental animals, Balb/c bone marrow and splenocytes are incubated for 5 hrs with C57BL/6 MHC proteins, peptides, or exosomes containing C57BL/6 allo-antigens, to activate any allo-reactive Balb/c T cells in the graft, and then treated in a Petri dish with PUVA (200 ng/mL 8-MOP, 0.1 J/cm² UVA) to inactivate these allo-reactive Balb/c T cells.
3 The prepared tissues are transplanted into the irradiated tumor-bearing recipient.
4 The recipient is then monitored for bone marrow engraftment (survival of graft recipient past 14 days, later confirmed by blood analysis), and signs of GvHD.

### Results

In such an experiment, the allogeneic control mice are expected to engraft well but develop severe GvHD that is uniformly lethal ˜day 25 (expected, as graft is a full mismatch), while the allogeneic PUVA mice engraft equally well and show reduced signs of GvHD and/or improved survival.

### Experiment 5—Reducing Allo-Reactive T Cells Using Proteins, Peptides or Exosomes as Stimulator Molecules

1 Balb/c T cells or splenocytes (H2d MHC haplotype) are labeled with CFSE dye following standard protocols.
2 The labeled cells are then stimulated by culturing for 1-5 days with C57BL/6 (H2b MHC haplotype) MHC proteins, peptides, or exosomes containing C57BL/6 allo-antigens.
3 The response of Balb/c T cells to C57BL/6 allo-antigens is measured and quantified by monitoring by flow cytometry (FACS) the proliferation of Balb/c T cells (CFSE dye dilution with successive T cell divisions), and/or by measuring factors indicative of T cell activation (such as IL-2, IFNg, etc) in the culture supernatants.

The invention further relates to the following embodiments.
1. A method to selectively reduce immunogenicity in a transplant or a part thereof prior to transplantation, the method comprising the following steps:
   a) providing a transplant or a part thereof from a donor;
   b) providing a tissue sample from a recipient comprising cells capable of presenting antigens;
   c) combining the transplant or part thereof of step a) with the tissue sample of step b); and
   d) exposing the combination of step c) to a low dose of at least one apoptotic agent.
2. A method to selectively reduce immunogenicity in a transplant or a part thereof prior to transplantation, the method comprising the following steps:
   a) providing a transplant or a part thereof from a donor;
   b) providing a tissue sample from a recipient comprising cells capable of presenting antigens;
   c) exposing the tissue sample of step b) to at least one anti-proliferative agent, at least one apoptotic agent, lethal radiation or at least one cycle of freeze-thawing;
   d) combining the transplant or part thereof of step a) with the exposed tissue sample of step c); and
   e) exposing the combination of step d) to a low dose of at least one apoptotic agent.
3. The method according to 1, wherein the method further comprises step c1) of co-culturing the transplant or part thereof of step a) with the tissue sample of step b).
4. The method according to 2, wherein the method further comprises step d1) of co-culturing the transplant or part thereof of step a) with the exposed tissue sample of step c).
5. The method according to 3 or 4, wherein co-culturing is performed for at least 0.5 hours (h), 1 h, 2 h, 3 h, 4 h, 5 h or 6 h.
6. The method according to any of 1 to 5, wherein the transplant or part thereof of step a) is derived from stem cells, bone marrow, peripheral blood, white blood cells, leukocytes or umbilical cord blood.
7. The method according to any of 1 to 6, wherein the transplant or part thereof of step a) comprises T-cells.
8. The method according to any of 1 to 6, wherein the transplant or part thereof of step a) has been depleted of at least a portion of T-cells.
9. The method according to any of 1 to 8, wherein the transplant or part thereof of step a) is an allogeneic or haploidentical transplant.
10. The method according to any of 1 to 9, wherein the tissue sample of step b) is derived from blood and/or spleen of the recipient.
11. The method according to any of 1 to 10, wherein the cells capable of presenting antigens from the recipient of step b) are antigen presenting cells.
12. The method according to 10, wherein the antigen presenting cells are dendritic cells.
13. The method according to 11, wherein the dendritic cells have been obtained by plate passage of peripheral blood mononuclear cells (PBMCs) from the recipient.
14. The method according to any of 2 or 4 to 13, wherein the at least one anti-proliferative agent of step c) is mitomycin C.
15. The method according to any of 2 or 4 to 13, wherein the at least one apoptotic agent of step c) is the combination of a psoralen and UVA, riboflavin-phosphate and UVA and/or 5-aminolevulinic acid and light.
16. The method according to 15, wherein the psoralen is 8-MOP or amotosalen.
17. The method according to any of 2 or 4 to 13, wherein the lethal radiation of step c) is ultraviolet radiation, gamma radiation, electron radiation or X-rays.
18. The method according to any of 1 to 17, wherein the at least one apoptotic agent of step d) or step e) is the combination of a psoralen and UVA, riboflavin-phosphate and UVA and/or 5-aminolevulinic acid and light.
19. The method according to 18, wherein the psoralen is 8-MOP or amotosalen.
20. The method according to 19, wherein the psoralen is 8-MOP.
21. The method according to 20, wherein the dose of 8-MOP is equal to or below 200 ng/mL.
22. The method according to 18, wherein the dose of UVA is equal to or below 1 J/cm², 0.5 J/cm², 0.2 J/cm2 or 0.1 J/cm².
23. The method according to any of 1 to 22, wherein the recipient's immune system has not been treated with radiation or chemotherapy before the transplantation.
24. The method according to any of 1 to 23, wherein the donor and/or recipient are mammalian, preferably human.
25. Transplant or part thereof obtained by a method according to any of 1 to 24.
26. The transplant of 25, wherein the transplant has reduced immunogenicity.
27. The transplant of 25, wherein allo-reactive immune cells have been reduced or eliminated.
28. Transplant or part thereof according to 25 to 27 for use in a method of preventing or reducing graft versus host disease.
29. A method to selectively reduce immunogenicity in a transplant or a part thereof prior to transplantation, the method comprising the following steps:
   a) providing a transplant or a part thereof from the donor;
   b) providing at least one recipient antigen;
   c) combining the transplant or part thereof of step a) with the at least one recipient antigen of step b); and
   d) exposing the combination of step c) to a low dose of at least one apoptotic agent.
30. The method of 29, wherein the method further comprises step c1) of co-culturing the transplant or part thereof of step a) with the at least one recipient antigen of step b).
31. The method according to 30, wherein step c1) of co-culturing is performed for at least 0.5 h, 1 h, 2 h, 3 h, 4 h, 5 h or 6 h.
32. The method according to any of 29 to 31, wherein the transplant of step a) is derived from stem cells, bone marrow, peripheral blood, white blood cells, leukocytes or umbilical cord blood.
33. The method according to any of 29 to 32, wherein the transplant or part thereof of step a) comprises T-cells.
34. The method according to any of 29 to 32, wherein the transplant or part thereof of step a) has been depleted of T-cells.
35. The method according to any of 29 to 34, wherein the transplant or part thereof of step a) is an allogeneic or haploidentical transplant.
36. The method according to any of 29 to 35, wherein the at least one recipient antigen of step b) is a protein, peptide, MHC molecule or a fragment thereof, a peptide-MHC combination, a cell lysate or any combination thereof.
37. The method according to any of 29 to 36, wherein the at least one apoptotic agent of step d) is the combination of a psoralen and UVA, riboflavin-phosphate and UVA and/or 5-aminolevulinic acid and light.
38. The method according to 37, wherein the psoralen is 8-MOP or amotosalen.
39. The method according to 37 or 38, wherein the psoralen is 8-MOP.
40. The method according to 39, wherein the dose of 8-MOP is equal to or below 200 ng/mL.
41. The method according to 37, wherein the dose of UVA is equal to or below 1 J/cm², 0.5 J/cm², 0.2 J/cm² or 0.1 J/cm².
42. The method according to any of 29 to 41, wherein the recipient's immune system has not been treated with radiation or chemotherapy before the transplantation.
43. The method according to any of 29 to 42, wherein the donor and recipient are mammalian, preferably human.
44. Transplant or part thereof obtained by a method according to any of 29 to 43.
45. The transplant of 44, wherein the transplant has reduced immunogenicity.
46. The transplant of 44, wherein allo-reactive immune cells have been reduced or eliminated.
47. Transplant or part thereof according to 44 to 46 for use in a method of preventing or reducing graft versus host disease.
48. A method of preventing or reducing graft versus host disease in a subject in need thereof, the method comprising implanting the transplant or part thereof according to 25 to 27 into the subject.
49. A method of preventing or reducing graft versus host disease in a subject in need thereof, the method comprising implanting the transplant or part thereof according to 44 to 46 into the subject.
50. A composition comprising:
   (a) a transplant from a donor or a part thereof;
   (b) at least one recipient antigen; and
   (c) an apoptotic agent or anti-proliferative agent.
51. The composition according to 50, wherein the proliferative capacity of allo-reactive donor T cells in the transplant is reduced by at least 10%, 20%, 30%, 40%, 50%, 60%, 70%, 80%, 90%, 95%, 98% or more.
52. The composition according to 50 or 51, wherein the transplant or a part thereof has reduced immunogenicity compared to a transplant from a donor or a part thereof that has not been exposed to at least one recipient antigen and an apoptotic agent or an antiproliferative agent.

1. A method to selectively reduce immunogenicity in a transplant or a part thereof prior to transplantation, the method comprising the following steps:
a) providing a transplant or a part thereof from a donor;
b) providing a tissue sample from a recipient comprising cells capable of presenting antigens;
c) combining the transplant or part thereof of step a) with the tissue sample of step b); and
d) exposing the combination of step c) to a low dose of at least one apoptotic agent.

2. A method to selectively reduce immunogenicity in a transplant or a part thereof prior to transplantation, the method comprising the following steps:
a) providing a transplant or a part thereof from a donor;
b) providing a tissue sample from a recipient comprising cells capable of presenting antigens;
c) exposing the tissue sample of step b) to at least one anti-proliferative agent, at least one apoptotic agent, lethal radiation or at least one cycle of freeze-thawing;
d) combining the transplant or part thereof of step a) with the exposed tissue sample of step c); and
e) exposing the combination of step d) to a low dose of at least one apoptotic agent.

3. The method according to claim 1, wherein the method further comprises step c1) of co-culturing the transplant or part thereof of step a) with the tissue sample of step b).

4. The method according to claim 2, wherein the method further comprises step d1) of co-culturing the transplant or part thereof of step a) with the exposed tissue sample of step c).

5. The method according to claim 3 or 4, wherein co-culturing is performed for at least 0.5 hours (h), 1 h, 2 h, 3 h, 4 h, 5 h or 6 h.

6. The method according to any of the preceding claims, wherein the transplant or part thereof of step a) is derived from stem cells, bone marrow, peripheral blood, white blood cells, leukocytes or umbilical cord blood.

7. The method according to any of the preceding claims, wherein the transplant or part thereof of step a) comprises T-cells.

8. The method according to any of claims 1 to 6, wherein the transplant or part thereof of step a) has been depleted of at least a portion of T-cells.

9. The method according to any of the preceding claims, wherein the transplant or part thereof of step a) is an allogeneic or haploidentical transplant.

10. The method according to any of the preceding claims, wherein the tissue sample of step b) is derived from blood and/or spleen of the recipient.

11. The method according to any of the preceding claims, wherein the cells capable of presenting antigens from the recipient of step b) are antigen presenting cells.

12. The method according to claim 11, wherein the antigen presenting cells are dendritic cells.

13. The method according to claim 12, wherein the dendritic cells have been obtained by plate passage of peripheral blood mononuclear cells (PBMCs) from the recipient.

14. The method according to any of claim 2 or 4 to 13, wherein the at least one anti-proliferative agent of step c) is mitomycin C.

15. The method according to any of claim 2 or 4 to 13, wherein the at least one apoptotic agent of step c) is the combination of a psoralen and UVA, riboflavin-phosphate and UVA and/or 5-aminolevulinic acid and light.

16. The method according to claim 15, wherein the psoralen is 8-MOP or amotosalen.

17. The method according to any of claim 2 or 4 to 13, wherein the lethal radiation of step c) is ultraviolet radiation, gamma radiation, electron radiation or X-rays.

18. The method according to any of the preceding claims, wherein the at least one apoptotic agent of step d) or step e) is the combination of a psoralen and UVA, riboflavin-phosphate and UVA and/or 5-aminolevulinic acid and light.

19. The method according to claim 18, wherein the psoralen is 8-MOP or amotosalen.

20. The method according to claim 19, wherein the psoralen is 8-MOP.

21. The method according to claim 20, wherein the dose of 8-MOP is equal to or below 200 ng/mL.

22. The method according to any of claims 18 to 21, wherein the dose of UVA is equal to or below 1 J/cm², 0.5 J/cm², 0.2 J/cm² or 0.1 J/cm².

23. The method according to any of the preceding claims, wherein the recipient's immune system has not been treated with radiation or chemotherapy before the transplantation.

24. The method according to any of the preceding claims, wherein the donor and/or recipient are mammalian, preferably human.

25. Transplant or part thereof obtained by a method according to any of claims 1 to 24.

26. The transplant of claim 25, wherein the transplant has reduced immunogenicity.

27. The transplant of claim 25, wherein allo-reactive immune cells have been reduced or eliminated.

28. Transplant or part thereof according to claims 25 to 27 for use in a method of preventing or reducing graft versus host disease.

29. A method to selectively reduce immunogenicity in a transplant or a part thereof prior to transplantation, the method comprising the following steps:
a) providing a transplant or a part thereof from the donor;
b) providing at least one recipient antigen;
c) combining the transplant or part thereof of step a) with the at least one recipient antigen of step b); and
d) exposing the combination of step c) to a low dose of at least one apoptotic agent.

30. The method of claim 29, wherein the method further comprises step c1) of co-culturing the transplant or part thereof of step a) with the at least one recipient antigen of step b).

31. The method according to claim 30, wherein step c1) of co-culturing is performed for at least 0.5 h, 1 h, 2 h, 3 h, 4 h, 5 h or 6 h.

32. The method according to any of claims 29 to 31, wherein the transplant of step a) is derived from stem cells, bone marrow, peripheral blood, white blood cells, leukocytes or umbilical cord blood.

33. The method according to any of claims 29 to 32, wherein the transplant or part thereof of step a) comprises T-cells.

34. The method according to any of claims 29 to 32, wherein the transplant or part thereof of step a) has been depleted of T-cells.

35. The method according to any of claims 29 to 34, wherein the transplant or part thereof of step a) is an allogeneic or haploidentical transplant.

36. The method according to any of claims 29 to 35, wherein the at least one recipient antigen of step b) is a protein, peptide, MHC molecule or a fragment thereof, a peptide-MHC combination, a cell lysate or any combination thereof.

37. The method according to any of claims 29 to 36, wherein the at least one apoptotic agent of step d) is the combination of a psoralen and UVA, riboflavin-phosphate and UVA and/or 5-aminolevulinic acid and light.

38. The method according to claim 37, wherein the psoralen is 8-MOP or amotosalen.

39. The method according to claim 37 or 38, wherein the psoralen is 8-MOP.

40. The method according to claim 39, wherein the dose of 8-MOP is equal to or below 200 ng/mL.

41. The method according to any of claims 37 to 40, wherein the dose of UVA is equal to or below 1 J/cm², 0.5 J/cm², 0.2 J/cm² or 0.1 J/cm².

42. The method according to any of claims 29 to 41, wherein the recipient's immune system has not been treated with radiation or chemotherapy before the transplantation.

43. The method according to any of claims 29 to 42, wherein the donor and recipient are mammalian, preferably human.

44. The method according to any of the preceding claims, wherein the immunoreactivity caused by the transplant or part thereof is reduced.

45. Transplant or part thereof obtained by a method according to any of claims 29 to 44.

46. The transplant of claim 45, wherein the transplant has reduced immunogenicity.

47. The transplant of claim 45, wherein allo-reactive immune cells have been reduced or eliminated.

48. Transplant or part thereof according to claims 45 to 47 for use in a method of preventing or reducing graft versus host disease.

49. A method of preventing or reducing graft versus host disease in a subject in need thereof, the method comprising implanting the transplant or part thereof according to claims 25 to 27 into the subject.

50. A method of preventing or reducing graft versus host disease in a subject in need thereof, the method comprising implanting the transplant or part thereof according to claims 45 to 47 into the subject.

51. A composition comprising:
(a) a transplant from a donor or a part thereof;
(b) at least one recipient antigen; and
(c) an apoptotic agent or anti-proliferative agent.

52. The composition according to claim 51, wherein the proliferative capacity of allo-reactive donor T cells in the transplant is reduced by at least 10%, 20%, 30%, 40%, 50%, 60%, 70%, 80%, 90%, 95%, 98% or more.

53. The composition according to claim 51 or 52, wherein the transplant or a part thereof has reduced immunogenicity compared to a transplant from a donor or a part thereof that has not been exposed to at least one recipient antigen and an apoptotic agent or an antiproliferative agent.
